(19) United States
(12) Plant Patent Application Publication
(10) Pub. No.: US 2002/0184683 P1
(43) Pub. Date: Dec. 5, 2002

(54) Chrysanthemum plant named 'Gedi One Cen'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,908

(22) Filed: Jun. 4, 2001

(51) Int. Cl. A01H 5/00

(52) U.S. Cl. PLT/289

(57) ABSTRACT

A new and distinct Chrysanthemum plant variety characterized by a decorative type inflorescence, consistent flowering response to short days, blooming consistently after 49 days of short day length, free branching habit, and compact growth habit.

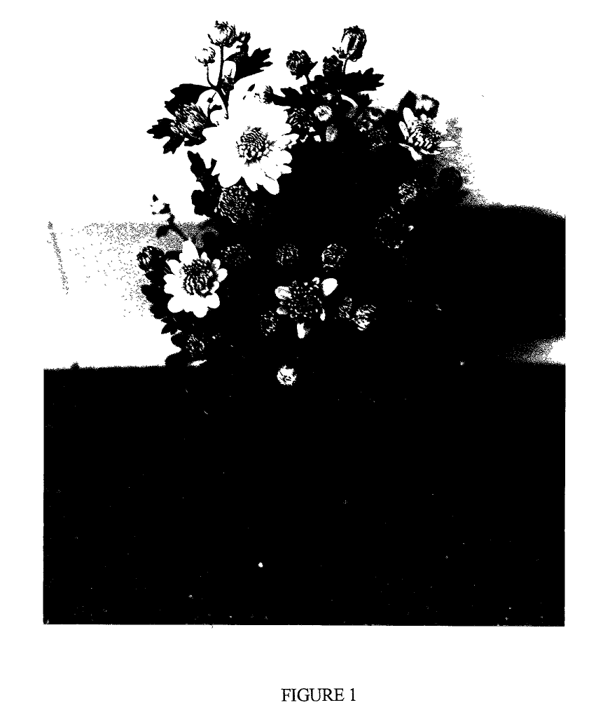

### BACKGROUND OF THE INVENTION

The present invention relates to a new and distinct cultivar of Chrysanthemum plant, botanically known as Dendranthema morifolium and herein after referred to by the cultivar name ‘GEDI ONE CEN.’

This new cultivar originated from hybridization of a female parent, cultivar ‘Bronia’(unpatented in the United States), and a male parent, cultivar ‘Cobra’ (U.S. Plant Pat. No. 10,219). The new cultivar was discovered by the inventor in October 1996.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE CEN’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE CEN.’ These characteristics in combination distinguish ‘GEDI ONE CEN’ as a new and distinct Chrysanthemum:

1. Decorative type inflorescence;

2. Consistent flowering response to short days, blooming consistently after 49 days of short day length;

3. Free branching habit; and

4. Compact growth habit.

Plants of the new cultivar are similar to plants of the parent cultivar, ‘Bronia,’ in most horticultural characteristics, however plants of the new cultivar differed from plants of the cultivar ‘Bronia’ in the following characteristics:

1. The new cultivar naturally blooms 2 weeks earlier;

2. The new cultivar has blooms more uniformly; and

3. The new cultivar has more blooms per flowering branch.

Plants of the new cultivar are also similar to plants of the parent cultivar, ‘Cobra,’ in most horticultural characteristics, however plants of the new cultivar differed from plants of the cultivar ‘Cobra’ in the following characteristics:

1. The new cultivar has different ray floret color;

2. The new cultivar has a different flower type;

3. The new cultivar has more blooms per flowering branch; and

4. The new cultivar has a more flexible plant form.

The new cultivar can be compared to the Chrysanthemum cultivar ‘Aldo’ (disclosed in Plant Pat. No. 10,216). However plants of the new Chrysanthemum differed from the plants of the cultivar ‘Aldo’ in the following characteristics:

1. New cultivar has more ray florets;

2. New cultivar has a more round plant shape and greater branching;

3. New cultivar has a shorter internode length; and

4. New cultivar has a more flexible plant form.

### BRIEF DESCRIPTION OF THE PHOTOGRAPHS

The photograph shows a plant of ‘GEDI ONE CEN’ grown in a 4-inch container. A single cutting was used in the pot.

### DETAILED DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE CEN’

Commercial classification: Garden-type Chrysanthemum

Parentage: Hybridization of female parent ‘Bronia’ (unpatented in the United States) and male parent ‘Cobra’ (U.S. Plant Pat. No. 10,219)

### Propagation

Time to Rooting: 7 to 14 days at approximately 21° C.

Rooting habit: Fine, fibrous

### Plant

Growth Habit: Mounding herbaceous perennial

Height: Approximately 14.7 cm

Growth rate: Moderate

Branching Characteristics: Free Branching

Length of Lateral Branches: Approximately 10 cm

Number of leaves per lateral branch: Approximately 7

### Foliage

Leaf:

Arrangement.—Alternate single

Average length.—Approximately 3.9 cm

Average width.—Approximately 2.5 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—youngf oliage upper side: Near 147A young foliage under side: Near 147A mature foliage upper side: Near 147B mature foliage under side. Near 147B venation upper side: Near 147A venation under side: Near 147B

Petiole:

Average length.—Approximately 2.5 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the last week of September

Number of inflorescences per stem.—Approximately 7

Inflorescence diameter.—Approximately 3.5 cm

Inflorescence depth.—Approximately 1 cm

Inflorescence longevity on plant.—Approximately 26-29 days

Persistence.—Persistent

Ray Florets:

Texture.—Shiny

Average number per flower.—Approximately 154

Shape.—Oblanceolate

Margin.—Entire

Apex.—Obtuse

Length.—Approximately 1.4 cm

Width.—Approximately 0.5 cm

Color.—upper surface at first opening: Near 15A upper surface at maturity: Near 12A upper surface at fading: Near 12A under surface at first opening: Near 15B under surface at maturity: Near 12B under surface at fading: Near 12B

Disc florets:

Appearance.—Shiny

Texture.—Smooth

Average number per flower.—Approximately 15

Shape.—Cylindric

Apex.—Obtuse

Average length.—Approximately 0.3 cm

Average width.—Approximately 0.1 cm

Color.—at first opening: Near 7A at maturity: Near 7B at fading: Near 7C

Peduncle:

Length.—at terminal end (shortest): Approximately 2.4 cm at lateral end (longest): Approximately 4.2 cm

Strength.—Moderate

Color.—Near 147B

Habit.—Erect

Inflorescence bud:

Length.—Approximately 0.9 cm

Diameter.—Approximately 0.6 cm

Form.—Globular

Color.—Near 167A

Sepals:

Appearance.—Matte

Texture.—Lightly pubescent

Number.—Approximately 40

Shape.—Ovate

Margin.—Entire

Apex.—Acute

Color.—upper side: Near 137A under side: Near 137C

### Reproductive Organs

Ray Florets:

Pistillate.—Number of pistils per flower: 1 Stigma shape: 2 branched Stigma color. Near 7A Style color: Near 154C Style length: Approximately 0.4 cm

Disc Florets:

Pistillate.—Number of pistils per flower: 1 Stigma shape: Cylindric Stigma color: Near 17A Style Color: Near 2D Style length: Approximately 0.3 cm

Staminate.—Number of stamens per flower: Approximately 5 Anther shape: Tubular Anther color: Near 15C Pollen color: Near 16A

### Other Characteristics

Disease Resistance: Neither resistance nor susceptibility observed

What is claimed is:

1. A new and distinct variety of Chrysanthemum plant named ‘GEDI ONE CEN’ as herein illustrated and described.
